(19) United States
(12) Patent Application Publication
(10) Pub. No.: US 2022/0008274 A1
(43) Pub. Date: Jan. 13, 2022

(54) DISPOSABLE PATIENT ISOLATION HOOD

(71) Applicants: Yu-Lin Shih, Taipei City (TW); Chin-Chuan Shih, Taipei City (TW); Yu-Chieh Shih, Taipei City (TW)

(72) Inventors: Yu-Lin Shih, Taipei City (TW); Chin-Chuan Shih, Taipei City (TW); Yu-Chieh Shih, Taipei City (TW)

(21) Appl. No.: 17/075,119

(22) Filed: Oct. 20, 2020

(30) Foreign Application Priority Data
Jul. 13, 2020 (TW) 109123567

(51) Int. Cl. A61G 10/00; A61B 90/40

(52) U.S. Cl. CPC A61G 10/005; A61B 90/40

(57) ABSTRACT

A disposable patient isolation hood is provided and includes a frame and an isolation element, wherein the frame includes a base and a support bridge, and two ends of the support bridge are respectively connected to the first end portion and the second end portion of the base. The isolation element is flexible, and at least a part thereof is made of a transparent material. The isolation element is arranged to cover the support bridge to form a space that can encompass at least a part of the subject in need. The disposable patient isolation hood is easy to carry and has high mobility, so that medical practitioners can be isolated and protected during medical treatment of patients even in the absence of medical resources or in emergency situations.

### BACKGROUND

### 1. Technical Field

The present disclosure generally relates to an isolation hood. More specifically, the present disclosure relates to a disposable patient isolation hood.

### 2. Description of Related Art

Medical practitioners are often faced with the risks of infection through droplets coming out of patients' mouths or noses or other discharged body fluids during medical treatments of the patients in close contact. During the outbreak of infectious diseases, protection against this risk of infection for medical practitioners can be crucial.

Even though personal protective equipment, such as protective clothing or medical masks, can be provided to medical practitioners, in clinical practice, there are many temporary or emergency situations where access to sufficient or sound personal protective equipment are not available, for example, in places where resources are limited, or during a pandemic where there is a sudden shortage of medical supplies. For example, during the outbreak of the pandemic coronavirus (COVID-19) in 2020, shortages of medical equipment due to overloaded medical systems meant that many surgeries or medical treatments were being performed on patients without additional protective measures, putting the lives of health workers at risk from the disease.

Therefore, there is a need for a lightweight, portable, high-mobility, easy-to-use, low-cost and effective protective equipment that reduces the risk of healthcare workers from being infected by patients when carrying out medical treatments during a shortage of medical resources or under emergency situations.

### SUMMARY

In order to address the needs above, among others, the present disclosure provides a disposable isolation hood, which may include: a frame including a base and a support bridge, wherein the base includes a first end portion and a second end portion, and two ends of the support bridge are connected with the first end portion and the second end portion, respectively, and the support bridge is bent above the base; and an isolation element that is flexible with at least a portion of the isolation element being made of a transparent material, wherein the isolation element is disposed on the support bridge to form a space encompassing at least a portion of a subject in need thereof.

In an embodiment, the isolation element is secured on the support bridge through a fastener.

In the isolation hood of the present disclosure, the extending directions of the two ends of the support bridge are perpendicular to the extending direction of the first end portion and the extending direction of the second end portion, respectively. In an embodiment, the support bridge is bent to form an arc shape or at least an angle. In another embodiment, the angle formed by the bend of the support bridge is between about 90 degrees and 150 degrees.

In the isolation hood of the present disclosure, the frame may further include a support member connected with the base, and the support member forms a geometric shape with the base to stabilize the structure of the frame. In an embodiment, the geometric shape is a triangle or a quadrilateral. In another embodiment, when the geometric shape is a triangle, the length of the support member is the length of the longest side of the triangle.

In the isolation hood of the present disclosure, the base may further include a connecting member connected with the first end portion and the second end portion, and the length of the connecting member is 1 to 5 times, for example, 1 to 4 times, 1 to 3 times, 1 to 2 times, or 1 to 1.5 times, of the length of the first end portion and the second end portion.

In the isolation hood of the present disclosure, the isolation element may further include at least one aperture that allows all or a portion of a user to pass through to perform medical treatment on the subject.

In an embodiment, the support member and the base are integrally formed. In another embodiment, the frame is used after assembling the support member, the base and the support bridge. In yet another embodiment, the frame is formed by assembling the support member, the base and the support bridge via at least one fastener.

In an embodiment, the frame or the isolation element or both is/are made of a disposable material. In another embodiment, the disposable material for making the frame can be selected from a group consisting of wood, plywood, cardboard, plastic corrugated sheet, synthetic sheet, plastic sheet, fiber sheet, honeycomb sheet, melamine sheet, foam sheet or any combination thereof. In still another embodiment, the disposable material for making the isolation element can be selected from biocompatible polymers, such as polypropylene, polystyrene, polycarbonate, polyethylene, polyester, polypropylene, and/or any other biocompatible plastics in the form of a sheet or a film, or any combination thereof.

The isolation hood of the present disclosure is lightweight, portable, highly mobile, easy-to-use, low-cost and effective in providing protection that reduces the risk of users from being infected when carrying out medical treatments on highly infectious subjects during a shortage of medical resources or under emergency situations. In addition, the isolation hood of the present disclosure is disposable, thereby eliminating the need for cumbersome sanitizing procedures required for repetitively-used protective equipment and the possibility of cross contamination.

### BRIEF DESCRIPTION OF THE DRAWINGS

FIGS. 1A to 1C are schematic views of an isolation hood (FIG. 1A) in accordance with an embodiment of the present disclosure, including a combination of a frame (FIG. 1B) and an isolation element (FIG. 1C);

FIG. 2 is a schematic view of a frame of an isolation hood in accordance with the present disclosure before assembly;

FIGS. 3A and 3B are schematic views of isolation hoods in accordance with different embodiments of the present disclosure, which include support bridges that are bent to form angles;

FIG. 4 is a schematic view of an isolation hood in accordance with an embodiment of the present disclosure, which includes the combination of frame and isolation element shown in FIG. 3B; and

FIGS. 5A and 5B are schematic views of isolation hoods in accordance with different embodiments of the present disclosure, which include the combination of a plurality of frames and an isolation element.

### DETAILED DESCRIPTION

Implementations of the present disclosure are illustrated with different embodiments. Other advantages and technical effects of the present disclosure can be readily understood by one of ordinary skill in the art upon reading the disclosure provided herein. It should be noted that the structures, ratios, sizes shown in the drawings appended to this specification are provided solely for illustrating the content disclosed herein in order to facilitate understanding and readability of the present disclosure by those skilled in the art. They are not meant, in any ways, to limit the implementable scope of the present disclosure. Thus, any modifications, changes, or adaptations, without affecting the effects created and objectives achieved by the present disclosure, are to be construed as falling within the scope covered by the technical contents disclosed herein.

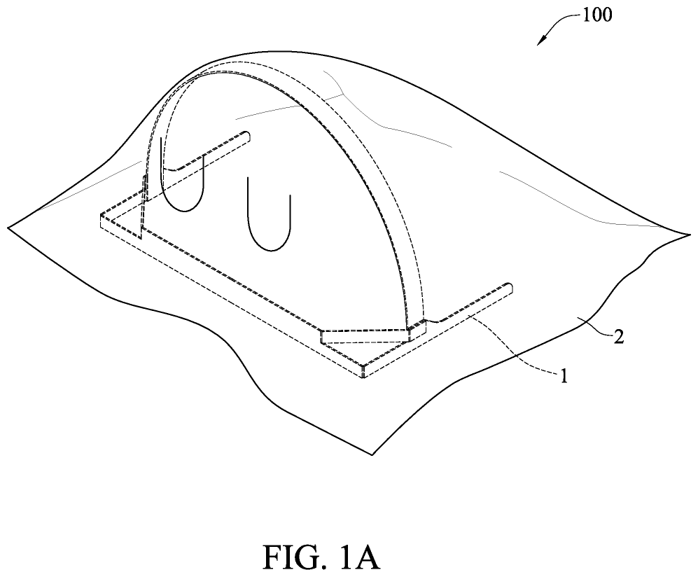

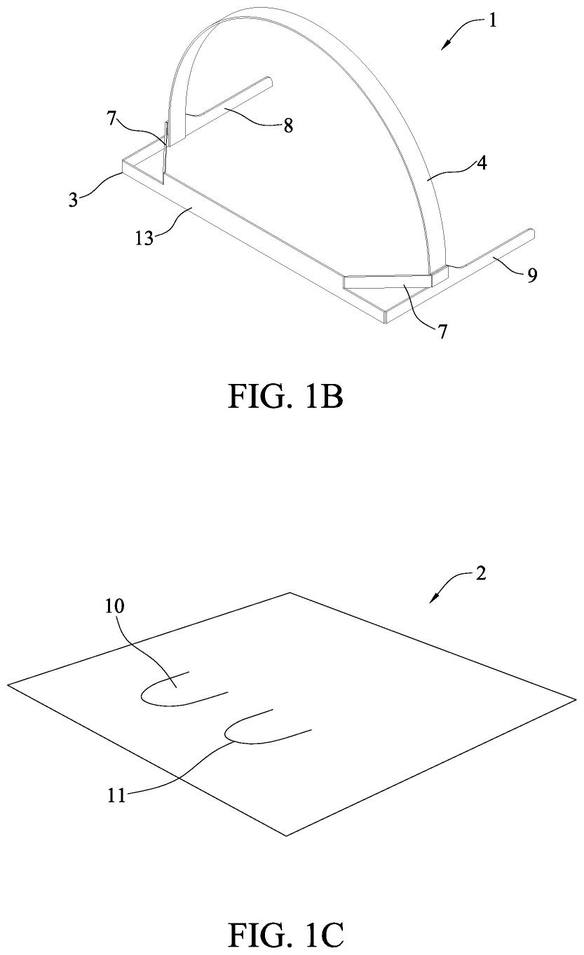

Referring to FIGS. 1A to 1C, a disposable patient isolation hood 100 is provided, which includes a frame 1 and an isolation element 2. The frame 1 includes a base 3 and a support bridge 4, wherein the base 3 includes a first end portion 8 and a second end portion 9, and two ends of the support bridge 4 are connected to the first end portion 8 and the second end portion 9, respectively. The bridge 4 is bent into an arc shape above the base 3.

In an embodiment, the isolation element 2 is disposed on the support bridge 4 and covers the entire frame 1 to form a space that can be used by a user or a subject in need. The space can encompass at least a portion of the subject; for example, when the subject is a person, the space can encompass the area above the individual's chest.

As shown in FIG. 1B, the extending direction of the two ends of the support bridge 4 can be perpendicular or substantially perpendicular to the extending direction of the first end portion 8 and the extending direction of the second end portion 9 of the base 3, respectively. In other words, the distance between the apex of the support bridge 4 and the horizontal plane formed by the base 3 is maximized. At this time, the space formed by the isolation element 2 covering the support bridge 4 is the largest, thereby providing the subject or the user with a maximum available space. In addition, the extending direction of the first end portion 8 of the base 3 can be parallel or substantially parallel to the extending direction of the second end portion 9 to maintain the stability of the frame 1 while providing the subject or the user with a maximum available space.

As shown again in FIG. 1B, the frame 1 includes a support member 7 connected to the base 3. The support member 7 can form any arbitrary geometric shape with the base 3 to improve the structural stability of the frame 1. For example, as illustrated, the geometric shape is a triangle (e.g., a right triangle) or a quadrilateral to improve the stability of the frame 1, and this geometric shape does not severely impede the available space to the user or the subject. When the geometric shape is a triangle, the length of the support member 7 can be the length of the longest side/hypotenuse of that triangle.

As can be seen again from FIG. 1B, the base 3 further includes a connecting member 13 connecting with the first end portion 8 and the second end portion 9. The lengths of the first end portion 8 and the second end portion 9 can be the same or different. The length of the first end portion 8 or the second end portion 9 can be defined as the distance between the vertex of an angle formed between the respective end and the connecting member 13 and the end point of that end. In an embodiment, the length of the connecting member 13 is 1 to 5 times of the length of the first end portion 8 or the second end portion 9, for example, 1 to 4 times, 1 to 3 times, 1 to 2 times, 1.5 to 2 times, including 1, 1.5, 2, 2.5, 3, 3.5, 4, 4.5 or 5 times, but the present disclosure is not limited as such, as long as the lengths of the first end portion 8, the second end portion 9 and the connecting member 13 can provide stability to the frame 1 while maintaining the space available to the subject or the user.

The material of the frame 1 can be selected from a material suitable for medical or other uses after being sanitized or cleaned, and can be a natural or synthetic disposable material, including but not limited to: wood, plywood, cardboard, plastic corrugated sheet, synthetic sheet, plastic sheet, fiber sheet, honeycomb sheet, melamine sheet, foam sheet or any combination thereof. In an embodiment, the materials of the base 3, the support member 7 and the support bridge 4 can be the same or different.

As shown in FIG. 1C, the isolation element 2 includes apertures 11 to allow all or a portion of a user to pass through to perform medical treatment on a subject. For example, as illustrated, the size of each of the apertures 11 is such that it allows the hands of a user to pass through, so that the user can perform operations in the space formed by the isolation element 2 (as shown in FIG. 1A). In an embodiment, the apertures 11 are formed by cutting the isolation element 2. For example, U-shaped cuts are formed at appropriate locations of the isolation element 2 to form apertures 11 with concealing flaps 10.

In an embodiment, a fastener (not shown) can be disposed at an appropriate location of the support bridge 4, in order to properly fasten the isolation element 2 and the support bridge 4, so that the isolation element 2 does not move from or slip off the support bridge 4 easily to impair the formed space, and the desired medical treatment can be carried out by the user successfully.

The isolation element 2 can be flexible and at least a portion of the isolation element 2 can be made by a transparent material to allow the user to perform medical treatment on or to observe the subject. For example, the isolation element 2 can include a transparent portion, a translucent portion, an opaque portion or any combination thereof. The material of the isolation element 2 can be selected from a material suitable for medical or other uses after being sanitized or cleaned, such as a natural or synthetic disposable material, including but not limited to: biocompatible polymers, such as polypropylene, polystyrene, polycarbonate, polyethylene, polyester, polypropylene, and/or any other biocompatible plastics in the form of a sheet or a film, or any combination thereof. The material of the isolation element 2 may further include a material with permeability, such as a permeable, semi-permeable and/or non-permeable plastic film or any combination thereof. The material used as the transparent portion of the isolation element 2 can be further anti-fog treated to provide a suitable environment for the user during precision operations.

In addition to providing the ability of isolating against sources of infection, the isolation element 2 may also have other functions, such as anti-fog, waterproof, windproof, cold protection, protection of individual privacy (for example, through the use of opaque materials) and/or ventilation or any combination thereof. Therefore, when using the isolation hood of the present disclosure, not only the risk of infection of the user or the subject can be reduced, comfort and safety can also be provided to the user or the subject.

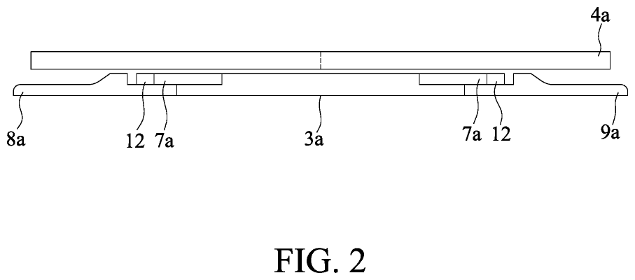

In an embodiment, the frame 1 can be used after assembly. In other words, before assembly, the base 3 and the support bridge 4 are separate components, and the support member 7, the first end portion 8 and the second end portion 9 of the base 3 can also be separate or partially connected components. Thus, the frame 1 is portable before assembly, and the various components can be assembled into the frame 1 for use. In addition, the various components of the base 3 can be an integral design. As shown in FIG. 2, a base 3a and a support bridge 4a before assembly are shown. The base 3a before assembly is an elongated sheet formed with a support member 7a, a first end portion 8a and a second end portion 9a after specific cuts; one end of which is respectively connected with a connecting member, while the unconnected ends are folded away. Similarly, the support bridge 4a before assembly is also an elongated sheet that can be folded away. As a result, the frame 1 is in a portable state before assembly.

The steps for preparing an integral base 3 are described as follows. A suitable elongated sheet is selected as the base 3a, and a portion is cut along a direction parallel to the longest central axis of the sheet to form the support member 7a; more specifically, the cut portion is not completely separated from the base 3a, and the cut portion is divided into the support member 7a and a joining portion 12 at an end. The joining portion 12 is used to fasten one end of the support member 7a that is not connected with the base 3a to the first end portion 8 or the second end portion 9 of the base 3a during assembly of the frame, such that the support member 7 and the base 3 form a geometric shape. In other words, after the aforementioned cutting, one end of the support member 7a is adjacent to the base 3a, while the other end of the support member 7a is adjacent to the joining portion 12. In addition, if needed, for example, for aesthetic reasons or improving stability of the base 3, additional cutting can be made to the non-essential portions of the base 3 and the support member 7.

Referring to FIG. 1B in conjunction with FIG. 2, during assembly of the base 3, the aforementioned joining portion 12 is joined to the base 3, such that the support member 7 and the base 3 form a geometric shape after being joined together, thereby completing the assembly of the integrally-formed support member 7 and the base 3 of the present disclosure. The location at which the joining portion 12 is joined with the base 3 can be adjusted depending on actual needs. In other words, the geometric shape formed by the support member 7 and the base 3 can be adjusted. Then, the two ends of the support bridge 4a are connected with the first end portion 8 and the second end portion 9 of the base 3, respectively, such that the support bridge 4a is arched above the base 3, serving as a support for the isolation element 2 when covering the frame 1, thereby completing the assembly of the isolation hood 100 of the present disclosure.

The junction (i.e., the joining portion 12) between the support member 7 and the base 3 and the junction between the base 3 and the support bridge 4 can be the same or different. For example, at least one end of the support bridge 4 can be adjacent to one of the vertices of the geometric shape formed by the support member 7 and the base 3, such as that shown in FIG. 1B. In addition, corresponding to the locations of these junctions and the location where the support bridge 4 touches the isolation element 2, fasteners can be respectively provided on the support member 7, the base 3, and the support bridge 4 depending on the needs to be used when joining them. The aforementioned joining can be achieved by chemical or physical joining methods, including, but not limited to, adhesives, magnetic fasteners, snap fasteners, and fastening clips, depending on the material used for the frame 1 or the isolation element 2.

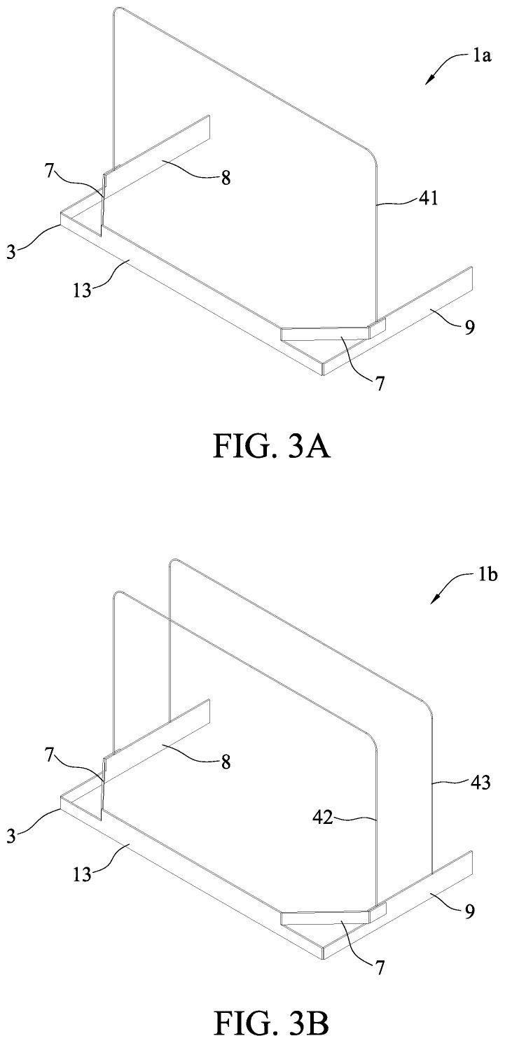

In an embodiment, the bend formed by the support bridge 4 above the base 3 may be arc-shaped or have an angle, as long as the bend is sufficient to support the isolation member 2 and constitute a space available for a user or a subject in need. As shown in FIGS. 3A and 3B, the frame 1a includes a support bridge 41, and the frame 1b includes support bridges 42 and 43. The bends formed by the support bridges 41, 42 and 43 on the base 3 are 90-degree angle or substantially about 90-degree angle or arc angle.

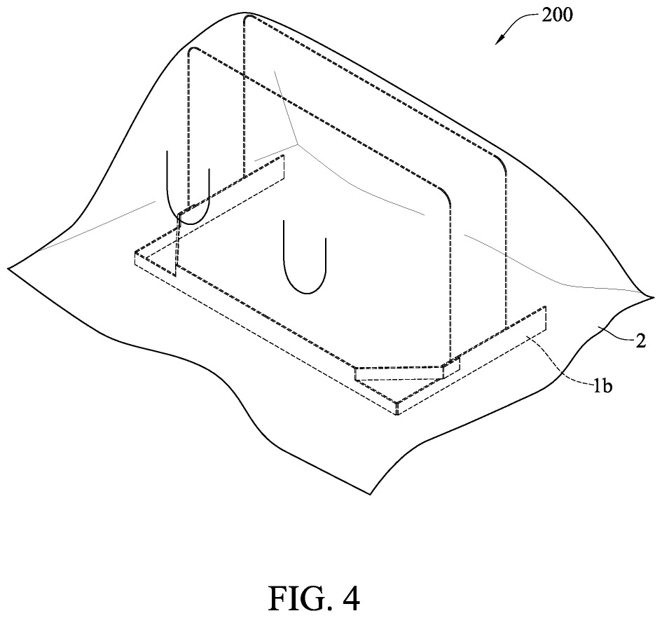

In the isolation hood of the present disclosure, if necessary, the frame may include a plurality of support bridges; that is, a plurality of support bridges may be provided on the base. As shown in FIG. 3B, the frame 1b includes the support bridges 42 and 43 connected to the base 3. FIG. 4 shows an isolation hood 200 when the frame 1b is used in combination with the isolation member 2.

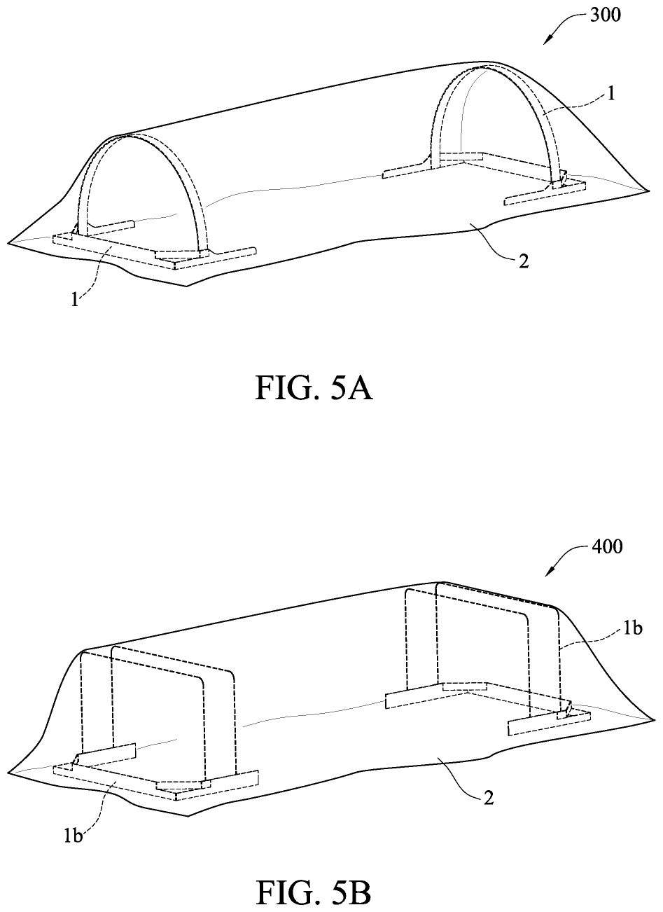

In the isolation hood of the present disclosure, if necessary, a plurality of frames may be included. As shown in FIGS. 5A and 5B, isolation hoods 300 and 400 include a plurality of frames 1 and frames 1b, respectively, and each includes an isolation element 2 that can completely cover the plurality of frames. In other embodiments of the present disclosure, the plurality of frames included may be the same or different; for example, the frame 1 can be used with the frame 1b. The space formed by the plurality of frames and the isolation element 2 is sufficient to cover a larger portion or the entire subject in need.

The isolation hood of the present disclosure is suitable for being placed on a hospital bed, and the configuration of the frame can improve the stability, so that the isolation hood can be stably placed on the hospital bed, and at the same time, the available space to a user or a subject can be maintained during medical treatment, so as to prevent the isolation hood from collapsing or falling from the hospital bed during medical treatment, thereby improving the protective function of the isolation hood of the present disclosure.

In addition, the isolation hood of the present disclosure can be further used in combination with a negative pressure device, so that the isolation hood of the present disclosure can further become a negative pressure protection equipment. More specifically, a negative pressure device having at least the functions of suction, filtering and/or sterilization can be provided in proximity to the isolation hood of the present disclosure to create a negative pressure environment in the space formed by the isolation member of the isolation hood of the present disclosure, and the gas in the space can be discharged after being concentrated, filtered and/or sterilized by the negative pressure device.

In summary, the isolation hood of the present disclosure may provide simplicity, portability, ease of use, low cost, effective isolation from infections, and high mobility, so that medical practitioners even in the absence of medical resources or in emergency situations can still be isolated and protected during medical treatment of patients, thereby reducing the risk of infection for healthcare workers. The isolation hood of the present disclosure can further provide anti-fog function to allow for the conditions when medical practitioners need precise operation. In addition, the isolation hood of the present disclosure can further provide patients with the features, such as waterproof, windproof, cold protection, personal privacy protection, ventilation, etc. In addition to providing isolation protection for users or subjects during use, the isolation hood of the present application can also offer comfort and safety.

While embodiments of the present disclosure have been disclosed in detail herein, it should be appreciated that the present disclosure is not limited thereto or thereby inasmuch as variations on the disclosure herein will be readily appreciated by those of ordinary skill in the art. The scope of the present disclosure shall be appreciated from the claims that follow.

What is claimed is:

1. A disposable isolation hood, comprising:
a frame including a base and a support bridge, wherein the base includes a first end portion and a second end portion, and wherein two ends of the support bridge are connected with the first end portion and the second end portion, respectively, and wherein the support bridge is bent above the base; and
an isolation element that is flexible with at least a portion of the isolation element being made of a transparent material,
wherein the isolation element is disposed on the support bridge to form a space encompassing at least a portion of a subject in need thereof.

2. The disposable isolation hood of claim 1, wherein extending directions of the two ends of the support bridge are perpendicular to an extending direction of the first end portion and an extending direction of the second end portion, respectively.

3. The disposable isolation hood of claim 1, further comprising a fastener for fastening the isolation element on the support bridge.

4. The disposable isolation hood of claim 1, wherein the frame further comprises a support member connected with the base.

5. The disposable isolation hood of claim 4, wherein the support member forms a geometric shape with the base to stabilize structure of the frame.

6. The disposable isolation hood of claim 5, wherein the geometric shape is at least one of a triangle or a quadrilateral.

7. The disposable isolation hood of claim 6, wherein the geometric shape is the triangle, and wherein a length of the support member is a length of a longest side of the triangle.

8. The disposable isolation hood of claim 1, wherein the support bridge is bent to form at least one of an arc shape or an angle.

9. The disposable isolation hood of claim 1, wherein the base further includes a connecting member connected with the first end portion and the second end portion.

10. The disposable isolation hood of claim 9, wherein a length of the connecting member is between one and five times a length of one of the first end portion and the second end portion.

11. The disposable isolation hood of claim 1, wherein the isolation element further includes at least one aperture for a user to pass through to perform medical treatment on the subject.

12. The disposable isolation hood of claim 4, wherein the support member and the base are integrally formed.

13. The disposable isolation hood of claim 12, wherein the frame is formed by assembling the support member, the base and the support bridge via at least one fastener.

14. The disposable isolation hood of claim 1, wherein at least one of the frame and the isolation element is made of a disposable material.
